(19) United States
(12) Patent Application Publication
(10) Pub. No.: US 2012/0241321 A1
(43) Pub. Date: Sep. 27, 2012

(54) POLYPEPTIDE MARKER FOR DIAGNOSING AND ASSESSING VASCULAR DISEASES

(76) Inventor: Harald Mischak, Sehnde (DE)

(21) Appl. No.: 13/395,757

(22) PCT Filed: Sep. 14, 2010
(86) PCT No.: PCT/EP2010/063475

§ 371 Date: May 22, 2012

(30) Foreign Application Priority Data
Sep. 14, 2009 (EP) 09170190.4

(51) Int. Cl. G01N 33/48; G01N 33/68; G01N 27/447; H01J 49/26; G01N 30/00

(52) U.S. Cl. 204/451; 250/282; 73/61.52; 73/61.43

(57) ABSTRACT

The invention relates to a method for diagnosing vascular diseases, comprising the step in which the presence, absence or amplitude of at least three polypeptide markers is determined in a urine sample, wherein the polypeptide markers are selected from the markers characterized in table 1 by values for the molecular masses and the migration time.

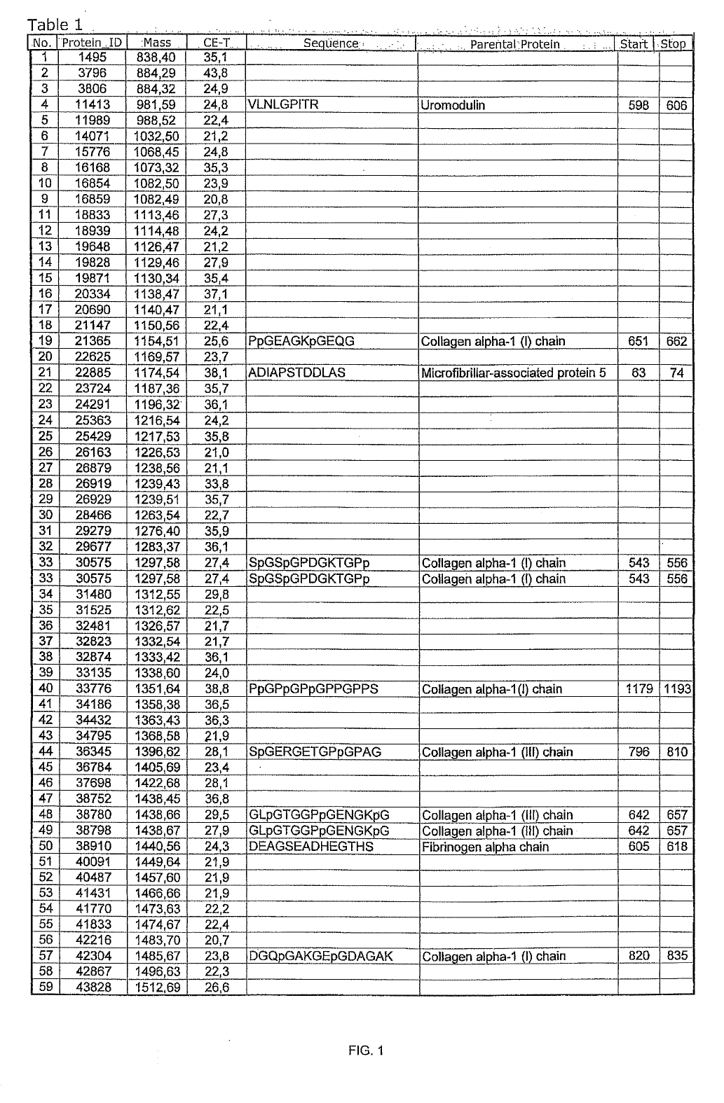

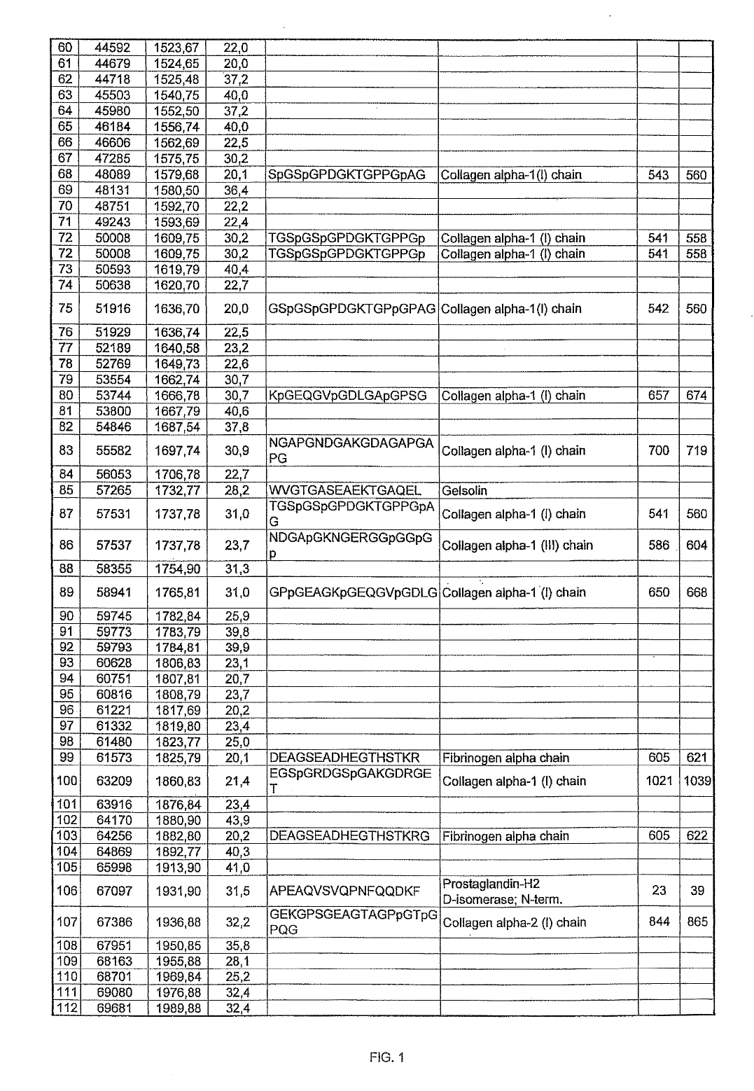

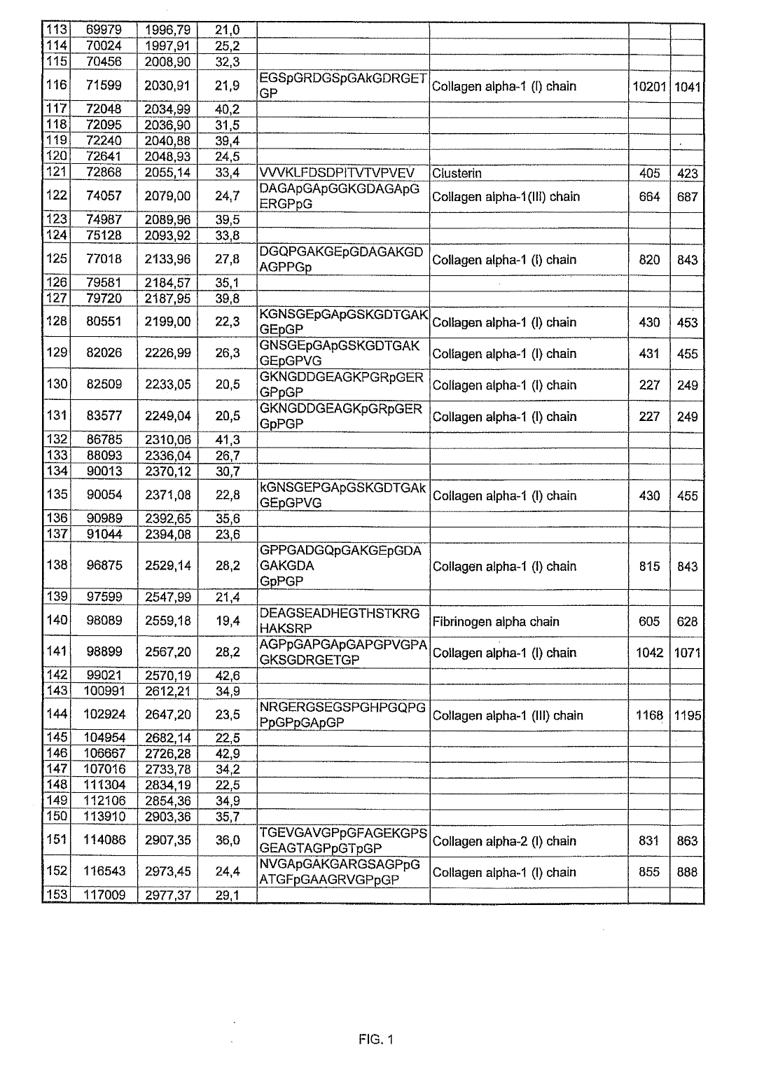

### DESCRIPTION OF THE INVENTION

The present invention relates to the use of the presence or absence of one or more peptide markers in a sample from a subject for the diagnosis and evaluation of severity of vascular diseases (VD) and to a method for the diagnosis and evaluation of such vascular disease, wherein the presence or absence of the peptide marker or markers is indicative of the severity of a VD.

Vascular diseases are diseases affecting the vessels of an organism and consequently organs such as the heart, brain, kidney etc. They include, for example, arteriosclerosis, disturbed circulation, hypertension and cardiac dysrhythmia.

Arteriosclerosis refers to the hardening of arteries by vascular deposits. Deposits of cholesterol crystals lead to the formation of inflammatory foci (atheromas) in which blood components, lipids, metabolic slags and lime salts tend to settle. So-called plaques are formed, which are two-dimensional scleroses, whereby the vascular wall becomes more rigid and narrower. The artery loses its elasticity and has difficulty in performing its task, i.e., the transport of blood from the heart into the individual regions of the body. Secondary diseases include, for example, angina pectoris, myocardial infarction, circulatory collapse, stroke. Disturbed circulation mostly affects the lower portion of the body, from the ventral aorta to the foot arteries, and leads to a reduction of blood flow and oxygen supply to the muscular tissue, which gradually becomes necrotic. In the last stage, ulcers form and occlude the vessels to such an extent that amputation becomes unavoidable. Hypertension has no definite cause; thus, the intake of medicaments or the excessive secretion of adrenal hormones can cause the blood pressure to surge. High blood pressures are also found in permanent stress, which results in angiospasms. Hypertension damages the vascular walls, so that there is a risk of tearing or obstruction. If the regularity of the heart beat is disturbed, the condition is referred to as cardiac dysrhythmia. The heart beat may be either too fast (tachycardia), too slow (bradycardia) or irregular (arrhythmia). Vascular diseases can be avoided by prevention, because they are also caused by an unhealthy and unnatural conduct of life. By a radical reversion of the way of living, arteriosclerosis in an early stage can be stalled, e.g., by reducing the blood pressure and blood lipid levels. The progress of vascular diseases can additionally be slowed down by medicamentous therapies (e.g., acetylsalicylic acid, beta receptor blockers, ACE inhibitors etc.). However, it is to be noted that damaged vessels are irreparable, and the process in an advanced stage is irreversible. Therefore, early detection of vascular diseases is particularly important.

In a coronary heart disease, the diagnosis of VD is effected at first indirectly by the evaluation of risk factors and by non-invasive examinations, such as measurement of blood pressure, resting and exercise electrocardiograms, and blood pictures for determining the lipid state (LDL cholesterol, HDL cholesterol, triglycerides), fasting blood glucose level and, if necessary, HbA1c. If such examinations yield the presence of high-risk characteristics, i.e., severe vascular events (death, myocardial infarction) are to be expected in the near future, a more exact diagnosis is made by means of invasive diagnostics, e.g., in the form of a catheter examination or coronary angiography. Thus, the heart and coronary vessels and other vessels are examined by means of a catheter or with an X-ray method. X-ray contrast media are used for a better visualization of the heart and vessels on the X-ray image. Indications of coronary angiography include a low or medium preliminary test probability while non-invasive diagnostics failed to provide reliable results, patients in whom non-invasive testing is not possible due to handicaps or diseases, and patients for whom exclusion with certainty of a suspected coronary heart disease is indispensable for work-related reasons (e.g., pilots, fire fighters). However, coronary angiography can be performed only if various complications, such as hyperthyroidism or allergy to contrast media, are excluded, in addition to the above mentioned preliminary examinations. In addition, since the contrast medium is secreted through the kidney, a sufficient renal function must be ensured, or for dialysis-dependent subjects, a dialysis must be performed always subsequent to the examination. Thus, it becomes clear that there is a need for a non-invasive possibility of an early and reliable diagnosis of vascular diseases.

Therefore, the object of the present invention is to provide processes and means for the diagnosis of vascular diseases.

This object is achieved by a process for the diagnosis of vascular diseases, comprising the step of determining the presence or absence or amplitude of at least three peptide markers in a urine sample, wherein said polypeptide markers are selected from the markers characterized in Table 1 by values for the molecular masses and migration times and in some cases their peptide sequence.

TABLE 1
Polypeptide markers

| | | Mass | CE-T | | | | |
|---|---|---|---|---|---|---|---|
| No. | Protein_ID | (Da) | (min) | Sequence | Parental Protein | Start | Stop |
| 1 | 1495 | 838.40 | 35.06 | | | | |
| 2 | 3796 | 884.29 | 43.81 | | | | |
| 3 | 3806 | 884.32 | 24.85 | | | | |
| 4 | 11413 | 981.59 | 24.80 | VLNLGPITR | Uromodulin | 598 | 606 |
| 5 | 11989 | 988.52 | 22.44 | | | | |
| 6 | 14071 | 1032.50 | 21.21 | | | | |
| 7 | 15776 | 1068.45 | 24.76 | | | | |
| 8 | 16168 | 1073.32 | 35.32 | | | | |
| 9 | 16859 | 1082.49 | 20.78 | | | | |
| 10 | 16854 | 1082.50 | 23.91 | | | | |
| 11 | 18833 | 1113.46 | 27.30 | | | | |
| 12 | 18939 | 1114.48 | 24.21 | | | | |
| 13 | 19648 | 1126.47 | 21.18 | | | | |
| 14 | 19828 | 1129.46 | 27.91 | | | | |
| 15 | 19871 | 1130.34 | 35.39 | | | | |
| 16 | 20334 | 1138.47 | 37.07 | | | | |
| 17 | 20690 | 1140.47 | 21.07 | | | | |
| 18 | 21147 | 1150.56 | 22.43 | | | | |
| 19 | 21365 | 1154.51 | 25.64 | PpGEAGKpGEQG | Collagen alpha-1 (I) chain | 651 | 662 |
| 20 | 22625 | 1169.57 | 23.71 | | | | |
| 21 | 22885 | 1174.54 | 38.13 | ADIAPSTDDLAS | Microfibrillar-associated | 63 | 74 |
| | | | | | protein 5 | | |
| 22 | 23724 | 1187.36 | 35.69 | | | | |
| 23 | 24291 | 1196.32 | 36.11 | | | | |
| 24 | 25363 | 1216.54 | 24.24 | | | | |
| 25 | 25429 | 1217.53 | 35.78 | | | | |
| 26 | 26163 | 1226.53 | 21.02 | | | | |
| 27 | 26879 | 1238.56 | 21.14 | | | | |
| 28 | 26919 | 1239.43 | 33.81 | | | | |
| 29 | 26929 | 1239.51 | 35.72 | | | | |
| 30 | 28466 | 1263.54 | 22.73 | | | | |
| 31 | 29279 | 1276.40 | 35.92 | | | | |
| 32 | 29677 | 1283.37 | 36.12 | | | | |
| 33 | 30575 | 1297.58 | 27.37 | SpGSpGPDGKTGPp | Collagen alpha-1 (I) chain | 543 | 556 |
| 34 | 31480 | 1312.55 | 29.77 | | | | |
| 35 | 31525 | 1312.62 | 22.45 | | | | |
| 36 | 32481 | 1326.57 | 21.67 | | | | |
| 37 | 32823 | 1332.54 | 21.74 | | | | |
| 38 | 32874 | 1333.42 | 36.11 | | | | |
| 39 | 33135 | 1338.60 | 23.99 | | | | |
| 40 | 33776 | 1351.64 | 38.76 | | | | |
| 41 | 34186 | 1358.38 | 36.46 | | | | |
| 42 | 34432 | 1363.43 | 36.34 | | | | |
| 43 | 34795 | 1368.58 | 21.90 | | | | |
| 44 | 36345 | 1396.62 | 28.12 | SpGERGETGPpGPAG | Collagen alpha-1 (III) chain | 796 | 810 |
| 45 | 36784 | 1405.69 | 23.42 | | | | |
| 46 | 37698 | 1422.68 | 28.14 | | | | |
| 47 | 38752 | 1438.45 | 36.76 | | | | |
| 48 | 38780 | 1438.66 | 29.52 | GLpGTGGPpGENGKpG | Collagen alpha-1 (III) chain | 642 | 657 |
| 49 | 38798 | 1438.67 | 27.88 | GLpGTGGPpGENGKpG | Collagen alpha-1 (III) chain | 642 | 657 |
| 50 | 38910 | 1440.56 | 24.30 | DEAGSEADHEGTHS | Fibrinogen alpha chain | 605 | 618 |
| 51 | 40091 | 1449.64 | 21.86 | | | | |
| 52 | 40487 | 1457.60 | 21.93 | | | | |
| 53 | 41431 | 1466.66 | 21.87 | | | | |
| 54 | 41770 | 1473.63 | 22.21 | | | | |
| 55 | 41833 | 1474.67 | 22.44 | | | | |
| 56 | 42216 | 1483.70 | 20.65 | | | | |
| 57 | 42304 | 1485.67 | 23.77 | DGQpGAKGEpGDAGAK | Collagen alpha-1 (I) chain | 820 | 835 |
| 58 | 42867 | 1496.63 | 22.34 | | | | |
| 59 | 43828 | 1512.69 | 26.62 | | | | |
| 60 | 44592 | 1523.67 | 21.97 | | | | |
| 61 | 44679 | 1524.65 | 20.03 | | | | |
| 62 | 44718 | 1525.48 | 37.16 | | | | |
| 63 | 45503 | 1540.75 | 39.98 | | | | |
| 64 | 45980 | 1552.50 | 37.21 | | | | |
| 65 | 46184 | 1556.74 | 40.03 | | | | |
| 66 | 46606 | 1562.69 | 22.46 | | | | |
| 67 | 47285 | 1575.75 | 30.20 | | | | |
| 68 | 48089 | 1579.68 | 20.06 | | | | |
| 69 | 48131 | 1580.50 | 36.39 | | | | |
| 70 | 48751 | 1592.70 | 22.18 | | | | |
| 71 | 49243 | 1593.69 | 22.38 | | | | |
| 72 | 50008 | 1609.75 | 30.20 | TGSpGSpGPDGKTGPPGp | Collagen alpha-1 (I) chain | 541 | 558 |
| 73 | 50593 | 1619.79 | 40.40 | | | | |
| 74 | 50638 | 1620.70 | 22.66 | | | | |
| 75 | 51916 | 1636.70 | 20.03 | | | | |
| 76 | 51929 | 1636.74 | 22.50 | | | | |
| 77 | 52189 | 1640.58 | 23.24 | | | | |
| 78 | 52769 | 1649.73 | 22.64 | | | | |
| 79 | 53554 | 1662.74 | 30.70 | | | | |
| 80 | 53744 | 1666.78 | 30.66 | KpGEQGVpGDLGApGPSG | Collagen alpha-1 (I) chain | 657 | 674 |
| 81 | 53800 | 1667.79 | 40.56 | | | | |
| 82 | 54846 | 1687.54 | 37.79 | | | | |
| 83 | 55582 | 1697.74 | 30.88 | NGAPGNDGAKGDAGAPGAPG | Collagen alpha-1 (I) chain | 700 | 719 |
| 84 | 56053 | 1706.78 | 22.69 | | | | |
| 85 | 57265 | 1732.77 | 28.18 | | | | |
| 86 | 57537 | 1737.78 | 23.73 | NDGApGKNGERGGpGGpGp | Collagen alpha-1 (III) chain | 586 | 604 |
| 87 | 57531 | 1737.78 | 31.00 | TGSpGSpGPDGKTGPPGpAG | Collagen alpha-1 (I) chain | 541 | 560 |
| 88 | 58355 | 1754.90 | 31.26 | | | | |
| 89 | 58941 | 1765.81 | 31.00 | GPpGEAGKpGEQGVpGDLG | Collagen alpha-1 (I) chain | 650 | 668 |
| 90 | 59745 | 1782.84 | 25.91 | | | | |
| 91 | 59773 | 1783.79 | 39.82 | | | | |
| 92 | 59793 | 1784.81 | 39.92 | | | | |
| 93 | 60628 | 1806.83 | 23.06 | | | | |
| 94 | 60751 | 1807.81 | 20.65 | | | | |
| 95 | 60816 | 1808.79 | 23.72 | | | | |
| 96 | 61221 | 1817.69 | 20.23 | | | | |
| 97 | 61332 | 1819.80 | 23.36 | | | | |
| 98 | 61480 | 1823.77 | 25.04 | | | | |
| 99 | 61573 | 1825.79 | 20.13 | DEAGSEADHEGTHSTKR | Fibrinogen alpha chain | 605 | 621 |
| 100 | 63209 | 1860.83 | 21.40 | EGSpGRDGSpGAKGDRGET | Collagen alpha-1 (I) chain | 1021 | 1039 |
| 101 | 63916 | 1876.84 | 23.38 | | | | |
| 102 | 64170 | 1880.90 | 43.91 | | | | |
| 103 | 64256 | 1882.80 | 20.24 | DEAGSEADHEGTHSTKRG | Fibrinogen alpha chain | 605 | 622 |
| 104 | 64869 | 1892.77 | 40.25 | | | | |
| 105 | 65998 | 1913.90 | 40.96 | | | | |
| 106 | 67097 | 1931.90 | 31.49 | APEAQVSVQPNFQQDKF | Prostaglandin-H2 | 23 | 39 |
| | | | | | D-isomerase; N-term. | | |
| 107 | 67386 | 1936.88 | 32.24 | GEKGPSGEAGTAGP- | Collagen alpha-2 (I) chain | 844 | 865 |
| | | | | pGTpGPQG | | | |
| 108 | 67951 | 1950.85 | 35.77 | | | | |
| 109 | 68163 | 1955.88 | 28.11 | | | | |
| 110 | 68701 | 1969.84 | 25.23 | | | | |
| 111 | 69080 | 1976.88 | 32.38 | | | | |
| 112 | 69681 | 1989.88 | 32.44 | | | | |
| 113 | 69979 | 1996.79 | 20.98 | | | | |
| 114 | 70024 | 1997.91 | 25.16 | | | | |
| 115 | 70456 | 2008.90 | 32.29 | | | | |
| 116 | 71599 | 2030.91 | 21.85 | | | | |
| 117 | 72048 | 2034.99 | 40.19 | | | | |
| 118 | 72095 | 2036.90 | 31.52 | | | | |
| 119 | 72240 | 2040.88 | 39.35 | | | | |
| 120 | 72641 | 2048.93 | 24.46 | | | | |
| 121 | 72868 | 2055.14 | 33.40 | VVVKLFDSDPITVTVPVEV | Clusterin | 405 | 423 |
| 122 | 74057 | 2079.00 | 24.68 | | | | |
| 123 | 74987 | 2089.96 | 39.52 | | | | |
| 124 | 75128 | 2093.92 | 33.78 | | | | |
| 125 | 77018 | 2133.96 | 27.77 | DGQPGAKGEpGDAGAKG- | Collagen alpha-1 (I) chain | 820 | 843 |
| | | | | DAGPPGp | | | |
| 126 | 79581 | 2184.57 | 35.08 | | | | |
| 127 | 79720 | 2187.95 | 39.78 | | | | |
| 128 | 80551 | 2199.00 | 22.31 | | | | |
| 129 | 82026 | 2226.99 | 26.28 | GNSGEpGApGSKGDTGAK- | Collagen alpha-1 (I) chain | 431 | 455 |
| | | | | GEpGPVG | | | |
| 130 | 82509 | 2233.05 | 20.51 | GKNGDDGEAGKPGRpGERGP | Collagen alpha-1 (I) chain | 227 | 249 |
| | | | | pGP | | | |
| 131 | 83577 | 2249.04 | 20.53 | GKNGDDGEAGKpGRpGERG | Collagen alpha-1 (I) chain | 227 | 249 |
| | | | | pPGP | | | |
| 132 | 86785 | 2310.06 | 41.31 | | | | |
| 133 | 88093 | 2336.04 | 26.66 | | | | |
| 134 | 90013 | 2370.12 | 30.74 | | | | |
| 135 | 90054 | 2371.08 | 22.79 | | | | |
| 136 | 90989 | 2392.65 | 35.59 | | | | |
| 137 | 91044 | 2394.08 | 23.64 | | | | |
| 138 | 96875 | 2529.14 | 28.25 | GPPGADGQpGAKGEpGDA- | Collagen alpha-1 (I) chain | 815 | 843 |
| | | | | GAKGDA | | | |
| | | | | GpPGP | | | |
| 139 | 97599 | 2547.99 | 21.44 | | | | |
| 140 | 98089 | 2559.18 | 19.41 | DEAGSEADHEGTHSTKRGHA | Fibrinogen alpha chain | 605 | 628 |
| | | | | KSRP | | | |
| 141 | 98899 | 2567.20 | 28.21 | | | | |
| 142 | 99021 | 2570.19 | 42.56 | | | | |
| 143 | 100991 | 2612.21 | 34.91 | | | | |
| 144 | 102924 | 2647.20 | 23.47 | | | | |
| 145 | 104954 | 2682.14 | 22.49 | | | | |
| 146 | 106667 | 2726.28 | 42.94 | | | | |
| 147 | 107016 | 2733.78 | 34.16 | | | | |
| 148 | 111304 | 2834.19 | 22.47 | | | | |
| 149 | 112106 | 2854.36 | 34.86 | | | | |
| 150 | 113910 | 2903.36 | 35.71 | | | | |
| 151 | 114086 | 2907.35 | 35.96 | | | | |
| 152 | 116543 | 2973.45 | 24.37 | | | | |
| 153 | 117009 | 2977.37 | 29.12 | | | | |

The evaluation of the polypeptides measured can be done on the basis of the presence or absence or amplitude of the markers taking the following limits into account:

Specificity is defined as the number of actually negative samples divided by the sum of the numbers of the actually negative and false positive samples. A specificity of 100% means that a test recognizes all healthy persons as being healthy, i.e., no healthy subject is identified as being ill. This says nothing about how reliably the test recognizes sick patients.

Sensitivity is defined as the number of actually positive samples divided by the sum of the numbers of the actually positive and false negative samples. A sensitivity of 100% means that the test recognizes all sick persons. This says nothing about how reliably the test recognizes healthy patients.

By the markers according to the invention, it is possible to achieve a specificity of at least 60%, preferably at least 70%, more preferably at least 80%, even more preferably at least 90% and most preferably at least 95% for vascular diseases.

By the markers according to the invention, it is possible to achieve a sensitivity of at least 60%, preferably at least 70%, more preferably at least 80%, even more preferably at least 90% and most preferably at least 95% for vascular diseases.

The analysis of the presence or absence or amplitude was based on the following values:

TABLE 2

| | | | | Frequency | Mean | Frequency | Mean | Regulation |
|---|---|---|---|---|---|---|---|---|
| No. | Protein_ID | Mass | CE−T | VE | VE | Control | Control | factor* |
| 1 | 1495 | 838.40 | 35.1 | 35% | 36 | 31% | 92 | −2.3 |
| 2 | 3796 | 884.29 | 43.8 | 24% | 25 | 46% | 110 | −8.5 |
| 3 | 3806 | 884.32 | 24.9 | 67% | 177 | 85% | 537 | −3.8 |
| 4 | 11413 | 981.59 | 24.8 | 78% | 279 | 82% | 787 | −3.0 |
| 5 | 11989 | 988.52 | 22.4 | 47% | 113 | 26% | 72 | 2.8 |
| 6 | 14071 | 1032.50 | 21.2 | 59% | 349 | 64% | 633 | −2.0 |
| 7 | 15776 | 1068.45 | 24.8 | 95% | 1716 | 69% | 2751 | −1.2 |
| 8 | 16168 | 1073.32 | 35.3 | 97% | 4627 | 97% | 8438 | −1.8 |
| 9 | 16859 | 1082.49 | 20.8 | 26% | 40 | 23% | 39 | 1.2 |
| 10 | 16854 | 1082.50 | 23.9 | 48% | 92 | 56% | 246 | −3.1 |
| 11 | 18833 | 1113.46 | 27.3 | 78% | 238 | 84% | 389 | −1.8 |
| 12 | 18939 | 1114.48 | 24.2 | 38% | 52 | 59% | 173 | −5.1 |
| 13 | 19648 | 1126.47 | 21.2 | 54% | 148 | 54% | 302 | −2.0 |
| 14 | 19828 | 1129.46 | 27.9 | 62% | 130 | 76% | 257 | −2.4 |
| 15 | 19871 | 1130.34 | 35.4 | 82% | 410 | 81% | 759 | −1.8 |
| 16 | 20334 | 1138.47 | 37.1 | 40% | 51 | 34% | 79 | −1.3 |
| 17 | 20690 | 1140.47 | 21.1 | 76% | 1218 | 81% | 2251 | −2.0 |
| 18 | 21147 | 1150.56 | 22.4 | 71% | 209 | 53% | 147 | 1.9 |
| 19 | 21365 | 1154.51 | 25.7 | 77% | 276 | 71% | 584 | −2.0 |
| 20 | 22625 | 1169.57 | 23.7 | 46% | 41 | 39% | 63 | −1.3 |
| 21 | 22885 | 1174.54 | 38.1 | 22% | 50 | 32% | 87 | −2.5 |
| 22 | 23724 | 1187.36 | 35.7 | 61% | 525 | 80% | 1082 | −2.7 |
| 23 | 24291 | 1196.32 | 36.1 | 98% | 18682 | 97% | 32361 | −1.7 |
| 24 | 25363 | 1216.54 | 24.2 | 89% | 738 | 80% | 1604 | −2.0 |
| 25 | 25429 | 1217.53 | 35.8 | 54% | 1454 | 27% | 854 | 3.4 |
| 26 | 26163 | 1226.53 | 21.0 | 66% | 330 | 76% | 586 | −2.0 |
| 27 | 26879 | 1238.56 | 21.1 | 17% | 46 | 19% | 47 | −1.1 |
| 28 | 26919 | 1239.43 | 33.8 | 44% | 176 | 20% | 81 | 4.8 |
| 29 | 26929 | 1239.51 | 35.7 | 26% | 142 | 48% | 614 | −8.0 |
| 30 | 28466 | 1263.54 | 22.7 | 58% | 208 | 81% | 601 | −4.0 |
| 31 | 29279 | 1276.40 | 35.9 | 91% | 3155 | 99% | 5853 | −2.0 |
| 32 | 29677 | 1283.37 | 36.1 | 89% | 464 | 89% | 638 | −1.4 |
| 33 | 30575 | 1297.58 | 27.4 | 45% | 662 | 81% | 4441 | −12.1 |
| 34 | 31480 | 1312.55 | 29.8 | 98% | 1603 | 94% | 2021 | −1.2 |
| 35 | 31525 | 1312.62 | 22.5 | 78% | 604 | 68% | 675 | −1.0 |
| 36 | 32481 | 1326.57 | 21.7 | 25% | 51 | 23% | 48 | 1.2 |
| 37 | 32823 | 1332.54 | 21.7 | 53% | 408 | 10% | 102 | 21.1 |
| 38 | 32874 | 1333.42 | 36.1 | 53% | 77 | 59% | 128 | −1.9 |
| 39 | 33135 | 1338.60 | 24.0 | 80% | 288 | 59% | 258 | 1.5 |
| 40 | 33776 | 1351.64 | 38.8 | 65% | 158 | 43% | 158 | 1.5 |
| 41 | 34186 | 1358.38 | 36.5 | 96% | 2051 | 96% | 2797 | −1.4 |
| 42 | 34432 | 1363.43 | 36.3 | 98% | 1828 | 99% | 3211 | −1.8 |
| 43 | 34795 | 1368.58 | 21.9 | 29% | 97 | 35% | 154 | −1.9 |
| 44 | 36345 | 1396.62 | 28.1 | 75% | 488 | 62% | 757 | −1.3 |
| 45 | 36784 | 1405.69 | 23.4 | 75% | 267 | 67% | 297 | −1.0 |
| 46 | 37698 | 1422.68 | 28.1 | 74% | 2053 | 81% | 3255 | −1.7 |
| 47 | 38752 | 1438.45 | 36.8 | 97% | 5564 | 99% | 8313 | −1.5 |
| 48 | 38780 | 1438.66 | 30.2 | 31% | 90 | 35% | 171 | −2.1 |
| 49 | 38798 | 1438.67 | 27.9 | 98% | 3225 | 95% | 6756 | −2.0 |
| 50 | 38910 | 1440.56 | 24.3 | 35% | 77 | 44% | 128 | −2.1 |
| 51 | 40091 | 1449.64 | 21.9 | 99% | 5122 | 97% | 6380 | −1.2 |
| 52 | 40487 | 1457.60 | 21.9 | 26% | 45 | 28% | 75 | −1.8 |
| 53 | 41431 | 1466.66 | 21.9 | 100% | 2801 | 98% | 3314 | −1.2 |
| 54 | 41770 | 1473.63 | 22.2 | 39% | 231 | 42% | 439 | −2.0 |
| 55 | 41833 | 1474.67 | 22.4 | 56% | 429 | 53% | 540 | −1.2 |
| 56 | 42216 | 1483.70 | 20.7 | 57% | 157 | 41% | 177 | −0.8 |
| 57 | 42304 | 1485.67 | 23.8 | 96% | 1111 | 88% | 1138 | −0.9 |
| 58 | 42867 | 1496.63 | 22.3 | 21% | 25 | 23% | 52 | −2.3 |
| 59 | 43828 | 1512.69 | 26.6 | 54% | 65 | 25% | 37 | 3.8 |
| 60 | 44592 | 1523.67 | 22.0 | 93% | 4861 | 96% | 7108 | −1.5 |
| 61 | 44679 | 1524.65 | 20.0 | 89% | 526 | 74% | 619 | −1.0 |
| 62 | 44718 | 1525.48 | 37.2 | 100% | 2505 | 99% | 3428 | −1.4 |
| 63 | 45503 | 1540.75 | 40.0 | 34% | 866 | 55% | 1217 | −2.3 |
| 64 | 45980 | 1552.50 | 37.2 | 95% | 2320 | 92% | 3635 | −1.5 |
| 65 | 46184 | 1556.74 | 40.0 | 40% | 320 | 49% | 428 | −1.6 |
| 66 | 46606 | 1562.69 | 22.5 | 80% | 1276 | 75% | 1421 | −1.0 |
| 67 | 47285 | 1575.75 | 30.2 | 57% | 148 | 35% | 826 | −3.4 |
| 68 | 48089 | 1579.68 | 20.1 | 99% | 8298 | 100% | 12271 | −1.5 |
| 69 | 48131 | 1580.50 | 36.4 | 23% | 158 | 17% | 171 | −0.8 |
| 70 | 48751 | 1592.70 | 22.2 | 66% | 351 | 73% | 518 | −1.6 |
| 71 | 49243 | 1593.69 | 22.4 | 36% | 97 | 43% | 125 | −1.5 |
| 72 | 50008 | 1609.75 | 30.2 | 63% | 528 | 54% | 661 | −1.1 |
| 73 | 50593 | 1619.79 | 40.4 | 56% | 118 | 22% | 51 | 5.8 |
| 74 | 50638 | 1620.70 | 22.7 | 25% | 84 | 48% | 210 | −4.8 |
| 75 | 51916 | 1636.70 | 20.0 | 84% | 856 | 87% | 1765 | −2.1 |
| 76 | 51929 | 1636.74 | 22.5 | 99% | 12206 | 99% | 14540 | −1.2 |
| 77 | 52189 | 1640.58 | 23.2 | 94% | 4862 | 96% | 7587 | −1.6 |
| 78 | 52769 | 1649.73 | 22.6 | 80% | 707 | 79% | 838 | −1.2 |
| 79 | 53554 | 1662.74 | 30.7 | 31% | 46 | 33% | 106 | −2.5 |
| 80 | 53744 | 1666.78 | 30.7 | 61% | 159 | 73% | 413 | −3.1 |
| 81 | 53800 | 1667.79 | 40.6 | 45% | 293 | 21% | 116 | 5.4 |
| 82 | 54846 | 1687.54 | 37.8 | 89% | 229 | 62% | 177 | 1.9 |
| 83 | 55582 | 1697.74 | 30.9 | 93% | 1081 | 90% | 1354 | −1.2 |
| 84 | 56053 | 1706.78 | 22.7 | 98% | 880 | 93% | 1357 | −1.5 |
| 85 | 57265 | 1732.77 | 28.2 | 79% | 2155 | 93% | 2915 | −1.6 |
| 86 | 57537 | 1737.78 | 23.7 | 100% | 5346 | 96% | 6769 | −1.2 |
| 87 | 57531 | 1737.78 | 31.0 | 88% | 2357 | 93% | 3345 | −1.5 |
| 88 | 58355 | 1754.90 | 31.3 | 88% | 5814 | 58% | 4585 | 1.9 |
| 89 | 58941 | 1765.81 | 31.0 | 85% | 1586 | 80% | 1622 | −1.0 |
| 90 | 59745 | 1782.84 | 25.9 | 62% | 407 | 58% | 268 | 1.6 |
| 91 | 59773 | 1783.79 | 39.8 | 55% | 219 | 60% | 274 | −1.4 |
| 92 | 59793 | 1784.81 | 39.9 | 42% | 228 | 25% | 119 | 3.2 |
| 93 | 60628 | 1806.83 | 23.1 | 70% | 207 | 66% | 235 | −1.1 |
| 94 | 60751 | 1807.81 | 20.7 | 95% | 1928 | 94% | 3116 | −1.6 |
| 95 | 60816 | 1808.79 | 23.7 | 45% | 128 | 51% | 190 | −1.7 |
| 96 | 61221 | 1817.69 | 20.2 | 97% | 3122 | 98% | 4734 | −1.5 |
| 97 | 61332 | 1819.80 | 23.4 | 100% | 4640 | 99% | 6251 | −1.3 |
| 98 | 61480 | 1823.77 | 25.0 | 36% | 109 | 40% | 120 | −1.2 |
| 99 | 61573 | 1825.79 | 20.1 | 94% | 1756 | 93% | 2406 | −1.4 |
| 100 | 63209 | 1860.83 | 21.4 | 50% | 430 | 75% | 1000 | −3.5 |
| 101 | 63916 | 1876.84 | 23.4 | 75% | 292 | 73% | 295 | −1.0 |
| 102 | 64170 | 1880.90 | 43.9 | 49% | 456 | 64% | 729 | −2.1 |
| 103 | 64256 | 1882.80 | 20.2 | 100% | 25031 | 100% | 36640 | −1.5 |
| 104 | 64869 | 1892.77 | 40.3 | 29% | 221 | 26% | 477 | −1.9 |
| 105 | 65998 | 1913.90 | 41.0 | 55% | 147 | 33% | 80 | 3.1 |
| 106 | 67097 | 1931.90 | 31.5 | 40% | 79 | 20% | 30 | 5.3 |
| 107 | 67386 | 1936.88 | 32.2 | 91% | 315 | 85% | 370 | −1.1 |
| 108 | 67951 | 1950.85 | 35.8 | 84% | 714 | 84% | 780 | −1.1 |
| 109 | 68163 | 1955.88 | 28.1 | 68% | 301 | 52% | 249 | 1.6 |
| 110 | 68701 | 1969.84 | 25.2 | 75% | 771 | 71% | 701 | 1.2 |
| 111 | 69080 | 1976.88 | 32.4 | 36% | 164 | 47% | 219 | −1.7 |
| 112 | 69681 | 1989.88 | 32.4 | 86% | 272 | 84% | 320 | −1.1 |
| 113 | 69979 | 1996.79 | 21.0 | 82% | 561 | 83% | 1253 | −2.3 |
| 114 | 70024 | 1997.91 | 25.2 | 65% | 208 | 26% | 56 | 9.2 |
| 115 | 70456 | 2008.90 | 32.3 | 24% | 360 | 27% | 250 | 1.3 |
| 116 | 71599 | 2030.91 | 21.9 | 42% | 118 | 16% | 39 | 7.9 |
| 117 | 72048 | 2034.99 | 40.2 | 43% | 72 | 48% | 126 | −2.0 |
| 118 | 72095 | 2036.90 | 31.5 | 47% | 51 | 21% | 28 | 4.0 |
| 119 | 72240 | 2040.88 | 39.4 | 25% | 49 | 16% | 27 | 2.9 |
| 120 | 72641 | 2048.93 | 24.5 | 96% | 1499 | 93% | 1409 | 1.1 |
| 121 | 72868 | 2055.14 | 33.4 | 30% | 195 | 16% | 111 | 3.3 |
| 122 | 74057 | 2079.00 | 24.7 | 55% | 170 | 49% | 182 | −1.0 |
| 123 | 74987 | 2089.96 | 39.5 | 50% | 141 | 33% | 100 | 2.1 |
| 124 | 75128 | 2093.92 | 33.8 | 46% | 115 | 31% | 77 | 2.2 |
| 125 | 77018 | 2133.96 | 27.8 | 96% | 4175 | 94% | 2918 | 1.5 |
| 126 | 79581 | 2184.57 | 35.1 | 82% | 570 | 69% | 564 | 1.2 |
| 127 | 79720 | 2187.95 | 39.8 | 95% | 1501 | 93% | 1451 | 1.1 |
| 128 | 80551 | 2199.00 | 22.3 | 46% | 81 | 24% | 60 | 2.6 |
| 129 | 82026 | 2226.99 | 26.3 | 74% | 10980 | 93% | 17324 | −2.0 |
| 130 | 82509 | 2233.04 | 20.5 | 78% | 413 | 52% | 344 | 1.8 |
| 131 | 83577 | 2249.04 | 20.5 | 64% | 307 | 49% | 323 | −0.8 |
| 132 | 86785 | 2310.06 | 41.3 | 63% | 155 | 58% | 191 | −1.1 |
| 133 | 88093 | 2336.04 | 26.7 | 48% | 82 | 9% | 10 | 42.0 |
| 134 | 90013 | 2370.12 | 30.7 | 23% | 39 | 19% | 34 | 1.4 |
| 135 | 90054 | 2371.08 | 22.8 | 66% | 182 | 36% | 66 | 5.1 |
| 136 | 90989 | 2392.65 | 35.6 | 25% | 179 | 23% | 140 | 1.4 |
| 137 | 91044 | 2394.08 | 23.6 | 42% | 143 | 18% | 50 | 6.6 |
| 138 | 96875 | 2529.14 | 28.3 | 45% | 111 | 12% | 19 | 21.7 |
| 139 | 97599 | 2547.99 | 21.4 | 65% | 289 | 37% | 223 | 2.3 |
| 140 | 98089 | 2559.18 | 19.4 | 60% | 1075 | 44% | 790 | 1.9 |
| 141 | 98899 | 2567.20 | 28.2 | 53% | 197 | 24% | 64 | 6.8 |
| 142 | 99021 | 2570.19 | 42.6 | 95% | 7702 | 82% | 5976 | 1.5 |
| 143 | 100991 | 2612.21 | 34.9 | 87% | 711 | 74% | 428 | 2.0 |
| 144 | 102924 | 2647.20 | 23.5 | 68% | 129 | 37% | 135 | −0.6 |
| 145 | 104954 | 2682.14 | 22.5 | 96% | 1032 | 93% | 1167 | −1.1 |
| 146 | 106667 | 2726.28 | 42.9 | 91% | 5136 | 77% | 3966 | 1.5 |
| 147 | 107016 | 2733.78 | 34.2 | 61% | 184 | 36% | 84 | 3.7 |
| 148 | 111304 | 2834.19 | 22.5 | 42% | 84 | 37% | 75 | 1.3 |
| 149 | 112106 | 2854.36 | 34.9 | 98% | 4323 | 85% | 3723 | 1.3 |
| 150 | 113910 | 2903.36 | 35.7 | 42% | 36 | 15% | 11 | 8.9 |
| 151 | 114086 | 2907.35 | 36.0 | 76% | 314 | 34% | 80 | 8.8 |
| 152 | 116543 | 2973.45 | 24.4 | 70% | 408 | 42% | 212 | 3.2 |
| 153 | 117009 | 2977.37 | 29.1 | 75% | 253 | 38% | 101 | 5.0 |

*If mean(VD) > mean(control): frequency(VD)*mean(VD)/frequency(control)*mean(control)
If mean(VD) < mean(control): -(frequency(control)*mean(control)/frequency(VD)*mean(VD))
Mass in daltons
CE time in minutes

The migration time is determined by capillary electrophoresis (CE), for example, as set forth in the Example under item 2. Thus, a glass capillary of 90 cm in length and with an inner diameter (ID) of 75 μm and an outer diameter (OD) of 360 μm is operated at a voltage of 30 kV. As the solvent for the sample, 30% methanol, 0.5% formic acid in water is used.

It is known that the CE migration times may vary. Nevertheless, the order in which the polypeptide markers are eluted is typically the same for any CE system employed. In order to balance the differences in the migration time, the system may be normalized using standards for which the migration times are known. These standards may be, for example, the polypeptides stated in the Examples (see the Example, item 3).

The characterization of the polypeptide markers shown in Table 1 was determined by means of capillary electrophoresis-mass spectrometry (CE-MS), a method which has been described in detail, for example, by Neuhoff et al. (Rapid Communications in mass spectrometry, 2004, Vol. 20, pp. 149-156). The variation of the molecular masses between individual measurements or between different mass spectrometers is relatively small, typically within a range of ±0.1%, preferably within a range of ±0.05%, more preferably within a range of ±0.03%, even more preferably within a range of ±0.01% or 0.005%.

The polypeptide markers according to the invention are proteins or peptides or degradation products of proteins or peptides. They may be chemically modified, for example, by posttranslational modifications, such as glycosylation, phosphorylation, alkylation or disulfide bridges, or by other reactions, for example, within the scope of the degradation. In addition, the polypeptide markers may also be chemically altered, for example, oxidized, within the scope of the purification of the samples. Proceeding from the parameters that determine the polypeptide markers (molecular weight and migration time), it is possible to identify the sequence of the corresponding polypeptides by methods known in the prior art.

The polypeptides according to the invention are used to diagnose vascular diseases. “Diagnosis” means the process of knowledge gaining by assigning symptoms or phenomena to a disease or injury. The presence or absence of a polypeptide marker can be measured by any method known in the prior art. Methods which may be known are exemplified below.

A polypeptide marker is considered present if its measured value is at least as high as its threshold value. If the measured value is lower, then the polypeptide marker is considered absent. The threshold value can be determined either by the sensitivity of the measuring method (detection limit) or empirically.

In the context of the present invention, the threshold value is considered to be exceeded preferably if the measured value of the sample for a certain molecular mass is at least twice as high as that of a blank sample (for example, only buffer or solvent). The polypeptide marker or markers is/are used in such a way that its/their presence or absence is measured, wherein the presence or absence is indicative of the vascular diseases. Thus, there are polypeptide markers which are typically present in subjects with vascular diseases, but occur less frequently or are absent in subjects with no vascular diseases. Further, there are polypeptide markers which are present in patients with vascular diseases, but are less frequently or not at all present in patients with no vascular diseases.

In addition or also alternatively to the frequency markers (determination of presence or absence), amplitude markers may also be used for diagnosis. Amplitude markers are used in such a way that the presence or absence is not critical, but the height of the signal (the amplitude) decides if the signal is present in both groups. Two normalization methods are possible to achieve comparability between differently concentrated samples or different measuring methods. In the first approach, all peptide signals of a sample are normalized to a total amplitude of 1 million counts. Therefore, the respective mean amplitudes of the individual markers are stated as parts per million (ppm).

In addition, it is possible to define further amplitude markers by an alternative normalization method: In this case, all peptide signals of one sample are scaled with a common normalization factor. Thus, a linear regression is formed between the peptide amplitudes of the individual samples and the reference values of all known polypeptides. The slope of the regression line just corresponds to the relative concentration and is used as a normalization factor for this sample.

The decision for a diagnosis is made as a function of how high the amplitude of the respective polypeptide markers in the patient sample is in comparison with the mean amplitudes in the control groups or the “ill” group. If the amplitude rather corresponds to the mean amplitudes of the “ill” group, the existence of a vascular disease is to be considered, and if it rather corresponds to the mean amplitudes of the control group, the non-existence of a vascular disease is to be considered. The distance between the measured value and the mean amplitude can be considered a probability of the sample's belonging to a certain group. Alternatively, the distance between the measured value and the mean amplitude may be considered a probability of the sample's belonging to a certain group.

A frequency marker is a variant of an amplitude marker in which the amplitude in some samples is so low that it is below the detectionlimit. It is possible to convert such frequency markers to amplitude markers by including the corresponding samples in which the marker is not found into the calculation of the amplitude with a very small amplitude, on the order of the detection limit.

The subject from which the sample in which the presence or absence of one or more polypeptide markers is determined is derived may be any subject which is capable of suffering from vascular diseases. Preferably, the subject is a mammal, and most preferably, it is a human.

In a preferred embodiment of the invention, not just three polypeptide markers, but a larger combination of polypeptide markers are used. By comparing a plurality of polypeptide markers, a bias in the overall result from a few individual deviations from the typical presence probability in single individuals can be reduced or avoided.

The sample in which the presence or absence of the peptide marker or markers according to the invention is measured may be any sample which is obtained from the body of the subject. The sample is a sample which has a polypeptide composition suitable for providing information about the state of the subject. For example, it may be blood, urine, synovial fluid, a tissue fluid, a body secretion, sweat, cerebrospinal fluid, lymph, intestinal, gastric or pancreatic juice, bile, lacrimal fluid, a tissue sample, sperm, vaginal fluid or a feces sample. Preferably, it is a liquid sample. In a preferred embodiment, the sample is a urine sample.

Urine samples can be taken as preferred in the prior art. Preferably, a midstream urine sample is used as said urine sample in the context of the present invention. For example, the urine sample may also be taken by means of a urination apparatus as described in WO 01/74275.

The presence or absence of a polypeptide marker in the sample may be determined by any method known in the prior art that is suitable for measuring polypeptide markers. Such methods are known to the skilled person. In principle, the presence or absence of a polypeptide marker can be determined by direct methods, such as mass spectrometry, or indirect methods, for example, by means of ligands.

If required or desirable, the sample from the subject, for example, the urine sample, may be pretreated by any suitable means and, for example, purified or separated before the presence or absence of the polypeptide marker or markers is measured. The treatment may comprise, for example, purification, separation, dilution or concentration. The methods may be, for example, centrifugation, filtration, ultrafiltration, dialysis, precipitation or chromatographic methods, such as affinity separation or separation by means of ion-exchange chromatography, electrophoretic separation, i.e., separation by different migration behaviors of electrically charged particles in solution upon application of an electric field. Particular examples thereof are gel electrophoresis, two-dimensional polyacryl-amide gel electrophoresis (2D-PAGE), capillary electrophoresis, metal affinity chromatography, immobilized metal affinity chromatography (IMAC), lectin-based affinity chromatography, liquid chromatography, high-performance liquid chromatography (HPLC), normal and reverse-phase HPLC, cation-exchange chromatography and selective binding to surfaces. All these methods are well known to the skilled person, and the skilled person will be able to select the method as a function of the sample employed and the method for determining the presence or absence of the polypeptide marker or markers.

In one embodiment of the invention, the sample, before being separated by capillary electrophoresis, is separated, purified by ultracentrifugation and/or divided by ultrafiltration into fractions which contain polypeptide markers of a particular molecular size.

Preferably, a mass-spectrometric method is used to determine the presence or absence of a polypeptide marker, wherein a purification or separation of the sample may be performed upstream from such method. As compared to the currently employed methods, mass-spectrometric analysis has the advantage that the concentration of many (>100) polypeptides of a sample can be determined by a single analysis. Any type of mass spectrometer may be employed. By means of mass spectrometry, it is possible to measure 10 fmol of a polypeptide marker, i.e., 0.1 ng of a 10 kD protein, as a matter of routine with a measuring accuracy of about ±0.01% in a complex mixture. In mass spectrometers, an ion-forming unit is coupled with a suitable analytic device. For example, electrospray-ionization (ESI) interfaces are mostly used to measure ions in liquid samples, whereas MALDI (matrix-assisted laser desorption/ionization) is used for measuring ions from a sample crystallized in a matrix. To analyze the ions formed, quadrupoles, ion traps or time-of-flight (TOF) analyzers may be used, for example.

In electrospray ionization (ESI), the molecules present in solution are atomized, inter alia, under the influence of high voltage (e.g., 1-8 kV), which forms charged droplets at first that become smaller from the evaporation of the solvent. Finally, so-called Coulomb explosions result in the formation of free ions, which can then be analyzed and detected.

In the analysis of the ions by means of TOF, a particular acceleration voltage is applied which confers an equal amount of kinetic energy to the ions. Thereafter, the time that the respective ions take to travel a particular drifting distance through the flying tube is measured very accurately. Since with equal amounts of kinetic energy, the velocity of the ions depends on their mass, the latter can thus be determined. TOF analyzers have a very high scanning speed and therefore reach a good resolution.

Preferred methods for the determination of the presence and absence of polypeptide markers include gas-phase ion spectrometry, such as laser desorption/ionization mass spectrometry, MALDI-TOF MS, SELDI-TOF MS (surface-enhanced laser desorption/ionization), LC MS (liquid chromatography/mass spectrometry), 2D-PAGE/MS and capillary electrophoresis-mass spectrometry (CE-MS). All the methods mentioned are known to the skilled person.

A particularly preferred method is CE-MS, in which capillary electrophoresis is coupled with mass spectrometry. This method has been described in some detail, for example, in the German Patent Application DE 10021737, in Kaiser et al. (J. Chromatogr A, 2003, Vol. 1013: 157-171, and Electrophoresis, 2004, 25: 2044-2055) and in Wittke et al. (J. Chromatogr. A, 2003, 1013: 173-181). The CE-MS technology allows to determine the presence of some hundreds of polypeptide markers of a sample simultaneously within a short time and in a small volume with high sensitivity. After a sample has been measured, a pattern of the measured polypeptide markers is prepared, and this pattern can be compared with reference patterns of a sick or healthy subjects. In most cases, it is sufficient to use a limited number of polypeptide markers for the diagnosis of UAS. A CE-MS method which includes CE coupled on-line to an ESI-TOF MS is further preferred.

For CE-MS, the use of volatile solvents is preferred, and it is best to work under essentially salt-free conditions. Examples of such solvents include acetonitrile, isopropanol, methanol and the like. The solvents can be diluted with water or a weak acid (e.g., 0.1% to 1% formic acid) in order to protonate the analyte, preferably the polypeptides.

By means of capillary electrophoresis, it is possible to separate molecules by their charge and size. Neutral particles will migrate at the speed of the electro-osmotic flow upon application of a current, while cations are accelerated towards the cathode, and anions are delayed. The advantage of the capillaries in electrophoresis resides in the favorable ratio of surface to volume, which enables a good dissipation of the Joule heat generated during the current flow. This in turn allows high voltages (usually up to 30 kV) to be applied and thus a high separating performance and short times of analysis.

In capillary electrophoresis, silica glass capillaries having inner diameters of typically from 50 to 75 μm are usually employed. The lengths employed are, for example, 30-100 cm. In addition, the separating capillaries are usually made of plastic-coated silica glass. The capillaries may be either untreated, i.e., expose their hydrophilic groups on the interior surface, or coated on the interior surface. A hydrophobic coating may be used to improve the resolution. In addition to the voltage, a pressure may also be applied, which typically is within a range of from 0 to 1 psi. The pressure may also be applied only during the separation or altered meanwhile.

In a preferred method for measuring polypeptide markers, the markers of the sample are separated by capillary electrophoresis, then directly ionized and transferred on-line into a coupled mass spectrometer for detection. In the method according to the invention, it is advantageous to use several polypeptide markers for diagnosis. The use of at least 5, 6, 8 or 10 markers is preferred. In one embodiment, 20 to 50 markers are used.

In order to determine the probability of the existence of a disease when several markers are used, statistic methods known to the skilled person may be used. For example, the Random Forests method described by Weissinger et al. (Kidney Int., 2004, 65: 2426-2434) may be used by using a computer program such as S-Plus, or the support vector machines as described in the same publication.

### EXAMPLE

### 1. Sample Preparation

For detecting the polypeptide markers for diagnosis, urine was employed. Urine was collected from healthy donors (control group) as well as from patients suffering from vascular diseases. For the subsequent CE-MS measurement, the proteins which are also contained in the urine of patients in an elevated concentration, such as albumin and immunoglobulins, had to be separated off by ultrafiltration. Thus, 700 μl of urine was collected and admixed with 700 μm of filtration buffer (2 M urea, 10 mM ammonia, 0.02% SDS). This 1.4 ml of sample volume was ultrafiltrated (20 kDa, Sartorius, Gottingen, Germany). The ultrafiltration was performed at 3000 rpm in a centrifuge until 1.1 ml of ultrafiltrate was obtained.

The 1.1 ml of filtrate obtained was then applied to a PD 10 column (Amersham Bioscience, Uppsala, Sweden) and eluted with 2.5 ml of 0.01% NH₄OH, and lyophilized. For the CE-MS measurement, the polypeptides were then resuspended with 20 μl of water (HPLC grade, Merck).

### 2. CE-MS Measurement

The CE-MS measurements were performed with a capillary electrophoresis system from Beckman Coulter (P/ACE MDQ System; Beckman Coulter Inc., Fullerton, Calif., USA) and an ESI-TOF mass spectrometer from Bruker (micro-TOF MS, Bruker Daltonik, Bremen, Germany). The CE capillaries were supplied by Beckman Coulter and had an ID/OD of 50/360 μm and a length of 90 cm. The mobile phase for the CE separation consisted of 20% acetonitrile and 0.25% formic acid in water. For the “sheath flow” on the MS, 30% isopropanol with 0.5% formic acid was used, here at a flow rate of 2 μl/min. The coupling of CE and MS was realized by a CE-ESI-MS Sprayer Kit (Agilent Technologies, Waldbronn, Germany). For injecting the sample, a pressure of from 1 to a maximum of 6 psi was applied, and the duration of the injection was 99 seconds. With these parameters, about 150 nl of the sample was injected into the capillary, which corresponds to about 10% of the capillary volume. A stacking technique was used to concentrate the sample in the capillary. Thus, before the sample was injected, a 1 M NH₃ solution was injected for 7 seconds (at 1 psi), and after the sample was injected, a 2 M formic acid solution was injected for 5 seconds. When the separation voltage (30 kV) was applied, the analytes were automatically concentrated between these solutions. The subsequent CE separation was performed with a pressure method: 40 minutes at 0 psi, then 0.1 psi for 2 min, 0.2 psi for 2 min, 0.3 psi for 2 min, 0.4 psi for 2 min, and finally 0.5 psi for 32 min. The total duration of a separation run was thus 80 minutes.

In order to obtain as good a signal intensity as possible on the side of the MS, the nebulizer gas was turned to the lowest possible value. The voltage applied to the spray needle for generating the electrospray was 3700-4100 V. The remaining settings at the mass spectrometer were optimized for peptide detection according to the manufacturer's instructions. The spectra were recorded over a mass range of m/z 400 to m/z 3000 and accumulated every 3 seconds.

### 3. Standards for the CE Measurement

For checking and standardizing the CE measurement, the following proteins or polypeptides which are characterized by the stated CE migration times were employed:

| Protein/polypeptide | Migration time |
|---|---|
| Aprotinin (SIGMA, Taufkirchen, DE, Cat. # A1153) | 19.3 min |
| Ribonuclease (SIGMA, Taufkirchen, DE, Cat. # R4875) | 19.55 min |
| Lysozyme (SIGMA, Taufkirchen, DE, Cat. # L7651) | 19.28 min |
| “REV”, Sequence: REVQSKIGYGRQIIS | 20.95 min |
| “ELM”, Sequence: ELMTGELPYSHINNRDQIIFMVGR | 23.49 min |
| “KINCON”, Sequence: TGSLPYSHIGSRDQIIFMVGR | 22.62 min |
| “GIVLY” Sequence: GIVLYELMTGELPYSHIN | 32.2 min |

The proteins/polypeptides were employed at a concentration of 10 pmol/μl each in water. “REV”, “ELM, “KINCON” and “GIVLY” are synthetic peptides.

In principle, it is known to the skilled person that slight variations of the migration times may occur in separations by capillary electrophoresis. However, under the conditions described, the order of migration will not change. For the skilled person who knows the stated masses and CE times, it is possible without difficulty to assign their own measurements to the polypeptide markers according to the invention. For example, he may proceed as follows: At first, he selects one of the polypeptides found in his measurement (peptide 1) and tries to find one or more identical masses within a time slot of the stated CE time (for example, ±5 min). If only one identical mass is found within this interval, the assignment is completed. If several matching masses are found, a decision about the assignment is still to be made. Thus, another peptide (peptide 2) from the measurement is selected, and it is tried to identify an appropriate polypeptide marker, again taking a corresponding time slot into account. Again, if several markers can be found with a corresponding mass, the most probable assignment is that in which there is a substantially linear relationship between the shift for peptide 1 and that for peptide 2. Depending on the complexity of the assignment problem, it suggests itself to the skilled person to optionally use further proteins from his sample for assignment, for example, ten proteins. Typically, the migration times are either extended or shortened by particular absolute values, or compressions or expansions of the whole course occur. However, comigrating peptides will also comigrate under such conditions.

In addition, the skilled person can make use of the migration patterns described by Zuerbig et al. in Electrophoresis 27 (2006), pp. 2111-2125. If he plots his measurement in the form of m/z versus migration time by means of a simple diagram (e.g., with MS Excel), the line patterns described also become visible. Now, a simple assignment of the individual polypeptides is possible by counting the lines. Other approaches of assignment are also possible. Basically, the skilled person could also use the peptides mentioned above as internal standards for assigning his CE measurements.

1. A process for diagnosing vascular diseases, comprising the step of determining the presence or absence or amplitude of at least three polypeptide markers in a urine sample, wherein said polypeptide markers are selected from the markers characterized in Table 1 by their molecular masses and migration times.

2. The process according to claim 1, wherein an evaluation of the determined presence or absence or amplitude of the markers is effected by means of the reference values stated in Table 2.

3. The process according to claim 1, wherein at least five, at least six, at least eight, at least ten, at least 20 or at least 50 polypeptide markers as defined in claim 1 are used.

4. The process according to claim 1, wherein said sample from a subject is a midstream urine sample.

5. The process according to claim 1, wherein capillary electrophoresis, HPLC, gas-phase ion spectrometry and/or mass spectrometry is used for detecting the presence or absence or amplitude of said polypeptide markers.

6. The process according to claim 1, wherein a capillary electrophoresis is performed before the molecular mass of said polypeptide markers is measured.

7. The process according to claim 1, wherein mass spectrometry is used for detecting the presence or absence of said polypeptide markers.

8. (canceled)

9. A method for the diagnosis of vascular diseases comprising the steps of:
a) separating a sample into at least five, preferably 10, subsamples;
b) analyzing at least five subsamples for determining the presence or absence or amplitude of at least three polypeptide markers in the sample, wherein said polypeptide markers are selected from the markers of Table 1, which are characterized by their molecular masses and migrations times (CE times).

10. The method according to claim 9, wherein at least 10 subsamples are measured.

11. The method according to claim 9 wherein the migration times are based on a 90 cm length glass capillary having an inner diameter (ID) of 50 μm at an applied voltage of 25 kV, wherein 20% acetonitrile, 0.25 M formic acid in water is used as the mobile solvent.

12. (canceled)

13. The process according to claim 1, wherein the migration times are based on a 90 cm length glass capillary having an inner diameter (ID) of 50 μm at an applied voltage of 25 kV, wherein 20% acetonitrile, 0.25 M formic acid in water is used as the mobile solvent.

14. The process according to claim 1, having a sensitivity that is at least 60% and a specificity that is at least 40%.

15. The method according to claim 9, having a sensitivity that is at least 60% and a specificity that is at least 40%.


SEQUENCE LISTING (31 sequences)

SEQ ID NO: 1 - protein, 9 aa, HOMO SAPIENS
VLNLGPITR

SEQ ID NO: 2 - protein, 10 aa, HOMO SAPIENS
PGEAGKGEQG

SEQ ID NO: 3 - protein, 12 aa, HOMO SAPIENS
ADIAPSTDDLAS

SEQ ID NO: 4 - protein, 11 aa, HOMO SAPIENS
SGSGPDGKTGP

SEQ ID NO: 5 - protein, 13 aa, HOMO SAPIENS
SGERGETGPGPAG

SEQ ID NO: 6 - protein, 13 aa, HOMO SAPIENS
GLGTGGPGENGKG

SEQ ID NO: 7 - protein, 13 aa, HOMO SAPIENS
GLGTGGPGENGKG

SEQ ID NO: 8 - protein, 14 aa, HOMO SAPIENS
DEAGSEADHEGTHS

SEQ ID NO: 9 - protein, 14 aa, HOMO SAPIENS
DGQGAKGEGDAGAK

SEQ ID NO: 10 - protein, 15 aa, HOMO SAPIENS
TGSGSGPDGKTGPPG

SEQ ID NO: 11 - protein, 15 aa, HOMO SAPIENS
KGEQGVGDLGAGPSG

SEQ ID NO: 12 - protein, 20 aa, HOMO SAPIENS
NGAPGNDGAKGDAGAPGAPG

SEQ ID NO: 13 - protein, 15 aa, HOMO SAPIENS
NDGAGKNGERGGGGG

SEQ ID NO: 14 - protein, 17 aa, HOMO SAPIENS
TGSGSGPDGKTGPPGAG

SEQ ID NO: 15 - protein, 16 aa, HOMO SAPIENS
GPGEAGKGEQGVGDLG

SEQ ID NO: 16 - protein, 17 aa, HOMO SAPIENS
DEAGSEADHEGTHSTKR

SEQ ID NO: 17 - protein, 17 aa, HOMO SAPIENS
EGSGRDGSGAKGDRGET

SEQ ID NO: 18 - protein, 18 aa, HOMO SAPIENS
DEAGSEADHEGTHSTKRG

SEQ ID NO: 19 - protein, 17 aa, HOMO SAPIENS
APEAQVSVQPNFQQDKF

SEQ ID NO: 20 - protein, 20 aa, HOMO SAPIENS
GEKGPSGEAGTAGPGTGPQG

SEQ ID NO: 21 - protein, 19 aa, HOMO SAPIENS
VVVKLFDSDPITVTVPVEV

SEQ ID NO: 22 - protein, 22 aa, HOMO SAPIENS
DGQPGAKGEGDAGAKGDAGPPG

SEQ ID NO: 23 - protein, 22 aa, HOMO SAPIENS
GNSGEGAGSKGDTGAKGEGPVG

SEQ ID NO: 24 - protein, 21 aa, HOMO SAPIENS
GKNGDDGEAGKPGRGERGPGP

SEQ ID NO: 25 - protein, 20 aa, HOMO SAPIENS
GKNGDDGEAGKGRGERGPGP

SEQ ID NO: 26 - protein, 26 aa, HOMO SAPIENS
GPPGADGQGAKGEGDAGAKGDAGPGP

SEQ ID NO: 27 - protein, 24 aa, HOMO SAPIENS
DEAGSEADHEGTHSTKRGHAKSRP

SEQ ID NO: 28 - protein, 15 aa, HOMO SAPIENS
REVQSKIGYGRQIIS

SEQ ID NO: 29 - protein, 24 aa, HOMO SAPIENS
ELMTGELPYSHINNRDQIIFMVGR

SEQ ID NO: 30 - protein, 21 aa, HOMO SAPIENS
TGSLPYSHIGSRDQIIFMVGR

SEQ ID NO: 31 - protein, 18 aa, HOMO SAPIENS
GIVLYELMTGELPYSHIN